(19) United States
(12) Patent Application Publication
(10) Pub. No.: US 2017/0362599 A1
(43) Pub. Date: Dec. 21, 2017

(54) FRONT-END ANALOG SIGNAL PROCESSING FOR CELLULAR COMPUTATION

(71) Applicant: Massachusetts Institute of Technology, Cambridge, MA (US)

(72) Inventors: Ramez Daniel, Somerville, MA (US); Timothy Kuan-Ta Lu, Cambridge, MA (US); Jacob Rosenblum Rubens, Cambridge, MA (US)

(73) Assignee: Massachusetts Institute of Technology, Cambridge, MA (US)

(21) Appl. No.: 15/535,804

(22) PCT Filed: Dec. 22, 2015
(86) PCT No.: PCT/US2015/067454

§ 371 Date: Jun. 14, 2017

(51) Int. Cl. C12N 15/63; C12Q 1/68; C12N 15/70

(52) U.S. Cl. CPC C12N 15/635; C12N 15/70; C12Q 1/6897

(57) ABSTRACT

Aspects of the present disclosure relate to analog signal processing circuits and methods for cellular computation.

### RELATED APPLICATION

This application claims the benefit under 35 U.S.C. §119(e) of U.S. provisional application No. 62/095,457, filed Dec. 22, 2014, which is incorporated by reference herein in its entirety.

### FEDERALLY SPONSORED RESEARCH

This invention was made with government support under Grant Nos. CCF1124247 and DGE1122374 awarded by the National Science Foundation. The government has certain rights in the invention.

### FIELD

Aspects of the present disclosure relate to the field of biosynthetic engineering.

### BACKGROUND

Biology uses a mixed signal approach to understand an environment and implement an appropriate response. This mixed signal approach is often a combination of analog and digital signal processing. To date, most work in gene circuit design has been focused on digital signal processing.

### SUMMARY

Provided herein, in some aspects, are gene circuits and methods for analog signal processing. One of the aims of synthetic biology is to leverage biochemistry to implement computation (e.g., cellular computation). For complex computations, it is beneficial to have sensors that can measure the concentration of molecules of interest over a wide-range of concentrations. Previous methods of developing such sensors have used negative feedback or positive feedback to control the expression of a transcription factor that binds the molecule of interest. The present disclosure demonstrates that mutations in transcription factor binding sites (e.g., promoters) can be used to implement wide-dynamic range sensing without feedback control of the transcription factor.

Analog signal processing circuits as provided herein were designed to express multiple transcription factors with positive and negative wide-dynamic range sensing. Further, analog signal processing circuits, in some embodiments, were designed to include a riboswitch responsive to a molecule (e.g., an input signal) over a wide-dynamic range to control the translation rate of an output protein. The behavior implemented by a circuit with the two wide-dynamic range sensors (e.g., transcription factor binding site mutation and riboswitch) in series is that of a power-law-based analog-multiplier. Analog signal processing circuits for implementing wide-dynamic range sensors permit fine-tuned control of gene expression and complex bimolecular-based sensing and logic (e.g., analog-to-digital logic).

Some aspects of the present disclosure provide analog signal processing circuits comprising (a) a first promoter operably linked to a nucleic acid encoding a regulatory protein responsive to an input signal, and (b) a second promoter responsive to the regulatory protein and operably linked to a nucleic acid encoding an output molecule.

In some embodiments, the promoter of (a) is a constitutively-active promoter.

In some embodiments, the promoter of (a) is responsive to the regulatory protein.

In some embodiments, the promoter of (a) comprises a modification that alters the binding affinity of a transcription factor for the promoter of (a), relative to a similar unmodified promoter. In some embodiments, the modification is a nucleic acid mutation.

In some embodiments, the promoter of (b) comprises a modification that alters the binding affinity of a transcription factor for the promoter of (b), relative to a similar unmodified promoter. In some embodiments, the modification is a nucleic acid mutation.

In some embodiments, (a) and (b) are on the same vector. In some embodiments, the vector is a low copy plasmid, a medium copy plasmid or a high copy plasmid.

In some embodiments, the promoter of (b) is activated when bound by the regulatory protein. In some embodiments, the promoter of (b) is repressed when bound by the regulatory protein.

In some embodiments, (b) further comprises a regulatory sequence that regulates production of the output molecule and is located between the second promoter and the nucleic acid encoding the output molecule. In some embodiments, the regulatory sequence regulates transcription or translation of the output molecule. In some embodiments, the regulatory sequence is a ribosomal binding site. In some embodiments, the regulatory sequence is a modified ribosomal binding site. For example, the regulatory sequence may be a modified ribosomal binding site comprising a modification that alters the binding affinity of a ribosome for the modified ribosomal binding site, relative to a similar unmodified ribosomal binding site. In some embodiments, the regulatory sequence is a riboswitch. For example, the riboswitch may be responsive to (e.g., regulated by) theophylline.

In some embodiments, the promoter of (b) is a plux promoter that comprises a modification that alters the binding affinity of a transcription factor for the plux promoter of (b), relative to a similar unmodified promoter. In some embodiments, the promoter of (a) is operably linked to a nucleic acid encoding a LuxR protein. In some embodiments, the promoter of (a) is a constitutively-active promoter. In some embodiments, the promoter of (a) is a plux promoter. In some embodiments, the plux promoter of (a) comprises a modification that alters the binding affinity of a transcription factor for the plux promoter of (a), relative to a similar unmodified promoter.

In some embodiments, the promoter of (b) is a pBAD promoter that comprises a modification that alters the binding affinity of a transcription factor for the pBAD promoter of (b), relative to a similar unmodified promoter. In some embodiments, the promoter of (a) is operably linked to a nucleic acid encoding an AraC protein. In some embodiments, the promoter of (a) is a constitutively-active promoter. In some embodiments, the promoter of (a) is a pBAD promoter. In some embodiments, the pBAD promoter of (a) comprises a modification that alters the binding affinity of a transcription factor for the pBAD promoter of (a), relative to a similar unmodified promoter.

In some embodiments, the output molecule of (b) is a fluorescent output molecule.

Some aspects of the present disclosure provide analog signal processing circuits comprising (a) a first constitutively-active promoter operably linked to a nucleic acid encoding a regulatory protein responsive to an input signal, and (b) a second promoter responsive to the regulatory protein and operably linked to a nucleic acid encoding an output molecule, wherein the second promoter comprises a modification that alters the binding affinity of the regulatory protein for the second promoter, relative to a similar unmodified promoter.

Some aspects of the present disclosure provide analog signal processing circuits comprising (a) a first promoter operably linked to a nucleic acid encoding a regulatory protein responsive to an input signal, wherein the first promoter is responsive to the regulatory protein and comprises a modification that alters the binding affinity of the regulatory protein for the first promoter, and (b) a second promoter responsive to the regulatory protein and operably linked to a nucleic acid encoding an output molecule.

Some aspects of the present disclosure provide cells and/or cell lysates that comprise one or more analog processing circuits as provided herein.

In some embodiments, a cell is a bacterial cell. In some embodiments, a bacterial cell is an Escherichia coli cell.

In some embodiments, a cell and/or cell lysate further comprises an input signal. In some embodiments, an input signal modulates activity of the of the regulatory protein. For example, an input signal may activate the regulatory protein.

In some embodiments, the input signal is a chemical input signal.

Some aspects of the present disclosure provide methods of analog signal processing in cells. The methods may comprise, for example, providing a cell or cell lysate that comprises an analog processing circuit as provided herein, contacting the cell with an input signal that modulates the regulatory protein, and detecting in the cell or cell lysate an expression level of the output molecule.

In some embodiments, methods further comprise contacting the cell or cell lysate with different concentrations of the input signal.

In some embodiments, methods further comprise quantifying levels of the output molecule.

In some embodiments, the cell is a bacterial cell. In some embodiments, a bacterial cell is an Escherichia coli cell.

These and other aspects of the present disclosure are described in more detail herein.

The invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting.

### BRIEF DESCRIPTION OF DRAWINGS

The accompanying drawings are not intended to be drawn to scale. For purposes of clarity, not every component may be labeled in every drawing.

FIG. 1A shows a schematic of an example of analog signal processing circuit configured as a positive-feedback (PF) loop with a wild-type plux promoter driving expression of LuxR (referred to as a regulatory protein, which, when activated by acyl-homoserine lactone (AHL), binds to and activates the plux promoter) on a low copy plasmid (LCP) and one of three different mutated plux promoters (plux3, plux28 and plux56) driving expression of GFP (referred to as an output molecule) on a high copy plasmid (HCP). The relative affinities of the mutated plux promoters for LuxR differ. This circuit configuration is referred to as a positive-feedback loop because LuxR regulates its own expression in addition to the expression of GFP. FIG. 1B shows a graph of GFP expression as a function of AHL concentration for analog signal processing circuits that contain a wild-type plux promoter driving expression of LuxR and one of the three different mutated plux promoters driving expression of GFP. The data shows that analog signal processing circuits configured as positive-feedback loops and containing mutated transcription factor binding sites (e.g., promoters) that alter transcription factor binding affinity yield a wide-dynamic range of behavior in response to AHL without fusion to GFP (output molecule).

FIG. 2A shows a schematic of another example of analog signal processing circuit configured as a positive-feedback loop with one of three different mutated plux promoters driving expression of LuxR on a low copy plasmid (LCP) and one of three different mutated plux promoters driving expression of GFP on a high copy plasmid (HCP). FIG. 2B shows a graph of GFP expression as a function of AHL concentration for analog signal processing circuits that contain one of three different mutated plux promoters driving expression of LuxR and one of the three different mutated plux promoters driving expression of GFP. As shown by the data represented in FIG. 1B, analog signal processing circuits configured as positive-feedback loops and containing mutated transcription factor binding sites (e.g., promoters) that alter transcription factor binding affinity yield a wide-dynamic range power-law behavior in response to AHL without fusion to GFP (output molecule).

FIG. 3A shows a schematic of an example of analog signal processing circuit configured as an open loop (OL) with one of three different mutated plux promoters driving transcription of GFP and a constitutively-active pLlacO promoter driving expression of LuxR, both promoters on a high copy plasmid (HCP). FIG. 3B shows a graph of GFP expression as a function of AHL concentration for analog signal processing circuits that contain mutated plux promoters (plux3, plux28 and plux56) driving expression of GFP. The data shows that analog signal processing circuits configured as open loops and containing mutated transcription factor binding sites (e.g., promoters) that alter transcription factor binding affinity yield a wide-dynamic range of behavior in response to AHL without fusion to GFP (output molecule).

FIG. 4A shows a schematic of an example of analog signal processing circuit configured as a positive-feedback (PF) loop having power-law behavior with one of three different mutated plux promoters driving expression of GFP and one of three different mutated plux promoters driving expression of LuxR on a high copy plasmid. FIG. 4B and FIG. 4C show graphs of GFP expression as a function of AHL concentration for analog signal processing circuits that contain one of the three different mutated plux promoters driving expression of GFP and one of the three different mutated plux promoters driving expression of LuxR. The data shows that analog signal processing circuits configured as positive-feedback loops and containing mutated transcription factor binding sites (e.g., promoters) that alter transcription factor binding affinity yield a wide-dynamic range of power-law behavior in response to AHL.

FIG. 5A shows a schematic of an example of analog signal processing circuit configured as an open loop (OL) on a low copy plasmid. LuxR is produced constitutively from the pLlacO promoter and GFP production controlled by a wild-type plux promoter. LuxR, activated by AHL, binds to the wild-type plux promoter and activates transcription of GFP. FIG. 5B shows a schematic of an example of analog signal processing circuit configured as a positive-feedback (PF) loop on a low copy plasmid. LuxR and GFP production are controlled by respective wild-type plux promoters. LuxR, activated by AHL, binds to the wild-type plux promoters and activates transcription of LuxR and GFP. FIG. 5C shows a schematic of an example of analog signal processing circuit configured as an open loop (OL) on a low copy plasmid. LuxR is produced constitutively from the pLlacO promoter and GFP production controlled by a mutated plux promoter (plux56 promoter). LuxR, activated by AHL, binds to the mutated plux promoter with reduced affinity relative to the wild-type plux promoter and activates transcription of GFP. FIG. 5D shows a schematic of an example of analog signal processing circuit configured as a positive-feedback (PF) loop on a low copy plasmid. LuxR and GFP production are controlled respectively by a mutated plux promoter (plux56) and a wild-type plux promoter. LuxR, activated by AHL, binds to the plux promoters, with lower affinity for the mutated plux promoter, and activates transcription of LuxR and GFP. FIG. 5E shows a graph of GFP expression as a function of AHL concentration for the circuits shown in FIGS. 5A-5D. The circuits in FIG. 5A and FIG. 5B yield digital-like signal processing behavior. The circuits in FIG. 5C and FIG. 5D yield analog-like signal processing behavior. FIG. 5F shows the sensitivity of circuits in FIG. 5A-4D calculated from the GFP expression data in FIG. 5E.

FIG. 6A shows the placement of a LuxR binding site between the −35 and −10 region of the plux promoter that enables LuxR to function as a repressor of gene expression. FIG. 6B the behavior of LuxR as an activator or repressor in affecting GFP expression.

FIG. 7A shows a schematic of an example of analog signal processing circuit configured as an open-loop (OL) with one of three different mutated plux promoters (pluxREP, pluxREP3, and pluxREP56) that, when activated, repress expression of GFP and a constitutively-active promoter driving expression of LuxR on a high copy plasmid. In the pluxREP promoters, the wild-type or mutated LuxR binding site is placed between the −35 and −10 region of the promoter as in FIG. 6A. FIG. 7B shows a graph of GFP expression as a function of AHL concentration for analog signal processing circuits that contain mutated pluxREP promoters (pluxREP, pluxREP3, and pluxREP56) driving expression of GFP and constitutively-active pLlacO driving expression of LuxR. Mutation with repressor location in the promoter yields a negative-slope wide-dynamic range of behavior. Without mutation with repressor location, the promoter yields a negative-slope with “digital” behavior.

FIG. 8A shows a schematic of an example of analog signal processing circuit configured as an open-loop (OL) on a low copy plasmid with a truncated pBAD promoter driving expression of GFP and a constitutively-active pLlacO promoter driving expression of araC protein. AraC, activated by arabinose, binds to the truncated pBAD promoter with wild-type binding site (pRD1) or mutated I1 and I2 sites (31-30) with lower affinity to araC to activate expression of GFP. FIG. 8B shows a graph of GFP expression as a function of arabinose concentration for analog signal processing circuits that contain truncated pBAD promoter with wild-type or mutated I1 and I2 araC-binding sites driving expression of GFP. The mutated binding sites enable wide-dynamic range sensing of arabinose.

FIG. 9A shows the analog signal processing circuit depicted in FIG. 3A having a riboswitch in the 5′ untranslated region (5′ UTR) of GFP. The riboswitch forms a hairpin in the mRNA of GFP that occludes the ribosome binding site that activates translation of GFP. Theophylline binds the riboswitch, causing it to change conformation and expose the ribosome binding site, which activates translation of GFP. FIG. 9B shows a graph of GFP expression as a function of Theophylline concentration at a constant concentration of AHL for analog signal processing circuits that contain one of three different mutated plux promoters driving expression of GFP and a riboswitch, as shown in FIG. 9A. Theophylline controls the translation rate of GFP and, thus, GFP expression with a wide-dynamic range of behavior. FIG. 9C shows how simultaneously altering the concentrations of AHL and theophylline enables wide-dynamic range control of transcription and translation and can output a wide-range of GFP expression levels. FIG. 9D shows in two dimensions the data from FIG. 9C.

### DETAILED DESCRIPTION

For analog gene circuits, it can be advantageous for the input to be processed over a dynamic range so that there is a significant range of input concentrations upon which to implement logic (e.g., analog-to-digital logic). The present disclosure provides gene circuits and methods for implementing wide-dynamic range behavior of gene circuits and to tune analog function.

Analog signal processing circuits of the present disclosure comprise promoters responsive to an input signal and operably linked to a nucleic acid encoding an output molecule. A “promoter” is a control region of a nucleic acid at which initiation and rate of transcription of the remainder of a nucleic acid are controlled. A promoter may also contain sub-regions at which regulatory proteins and molecules, such as transcription factors, bind. Promoters of the present disclosure may be constitutive, inducible, activatable, repressible, tissue-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid.

A promoter is considered “responsive” to an input signal if the input signal modulates the function of the promoter, indirectly or directly. In some embodiments, an input signal may positively modulate a promoter such that the promoter activates, or increases (e.g., by a certain percentage or degree), transcription of a nucleic acid to which it is operably linked. In some embodiments, by contrast, an input signal may negatively modulate a promoter such that the promoter is prevented from activating or inhibits, or decreases, transcription of a nucleic acid to which it is operably linked. An input signal may modulate the function of the promoter directly by binding to the promoter or by acting on the promoter without an intermediate signal. For example, the LuxR protein modulates the plux promoter by binding to a region of the plux promoter. Thus, the LuxR protein is herein considered an input signal that directly modulates the plux promoter. By contrast, an input signal is considered to modulate the function of a promoter indirectly if the input signal modulates the promoter via an intermediate signal. For example, acyl-homoserine-lactone (AHL) modulates (e.g., activates) the LuxR protein, which, in turn, modulates (e.g., activates) the plux promoter. Thus, AHL is herein considered an input signal that indirectly modulates the plux promoter.

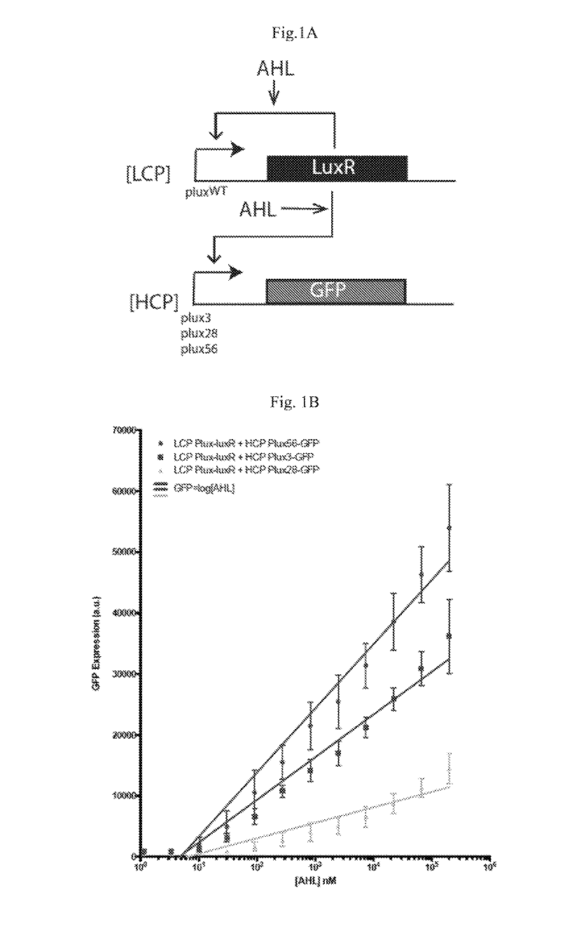

An “input signal” refers to any chemical (e.g., small molecule) or non-chemical (e.g., light or heat) signal in a cell, or to which the cell is exposed, that modulates, directly or indirectly, a component (e.g., a promoter) of an analog signal processing circuit. In some embodiments, an input signal is a biomolecule that modulates the function of a promoter (referred to as direct modulation), or is a signal that modulates a biomolecule, which then modulates the function of the promoter (referred to as indirect modulation). A “biomolecule” is any molecule that is produced in a live cell, e.g., endogenously or via recombinant-based expression. For example, with reference to FIG. 1A, AHL indirectly activates transcription of GFP via its activation of LuxR and subsequent binding of LuxR to the plux promoter. Thus, AHL is considered an input signal that indirectly modulates the plux promoter and, in turn, expression of GFP. Likewise, the LuxR protein is itself considered an input signal because it directly modulates transcription of GFP by binding to the plux promoter. In some embodiments, an input signal may be endogenous to a cell or a normally exogenous condition, compound or protein that contacts a promoter of an analog signal processing circuit in such a way as to be active in modulating (e.g., inducing or repressing) transcriptional activity from a promoter responsive to the input signal (e.g., an inducible promoter).

Examples of chemical input signals include, without limitation, signals extrinsic or intrinsic to a cell, such as amino acids and amino acid analogs, saccharides and polysaccharides, nucleic acids, protein transcriptional activators and repressors, cytokines, toxins, petroleum-based compounds, metal containing compounds, salts, ions, enzymes, enzyme substrates, enzyme substrate analogs, hormones and quorum-sensing molecules.

Examples of non-chemical input signals include, without limitation, changes in physiological conditions, such as changes in pH, light, temperature, radiation, osmotic pressure and saline gradients.

Promoters of the present disclosure that are responsive to an input signal and/or regulatory protein may be considered “inducible” promoters. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

A “positive-feedback promoter” refers to a promoter that is operably linked to a nucleic acid encoding a regulatory protein (e.g., transcription factor such as LuxR) that binds to that promoter to “self-regulate” expression of the regulatory protein. In some embodiments, a positive-feedback promoter is operably linked to a nucleic acid encoding a transcription factor that binds to the positive-feedback promoter to regulate its own expression. In some embodiments, positive-feedback promoters are modified (e.g., mutated) such that the affinity of the promoter for a particular regulatory protein is altered (e.g., reduced), relative to the affinity of the unmodified promoter for that same regulatory protein.

A “output promoter” refers to a promoter that is operably linked to a nucleic acid encoding an output molecule (e.g., GFP). In some embodiments, output promoters are responsive to a regulatory protein, such as, for example, a transcription factor. In some embodiments, output promoters are modified (e.g., mutated) such that the affinity of the promoter for a particular regulatory protein is altered (e.g., reduced), relative to the affinity of the unmodified promoter for that same regulatory protein.

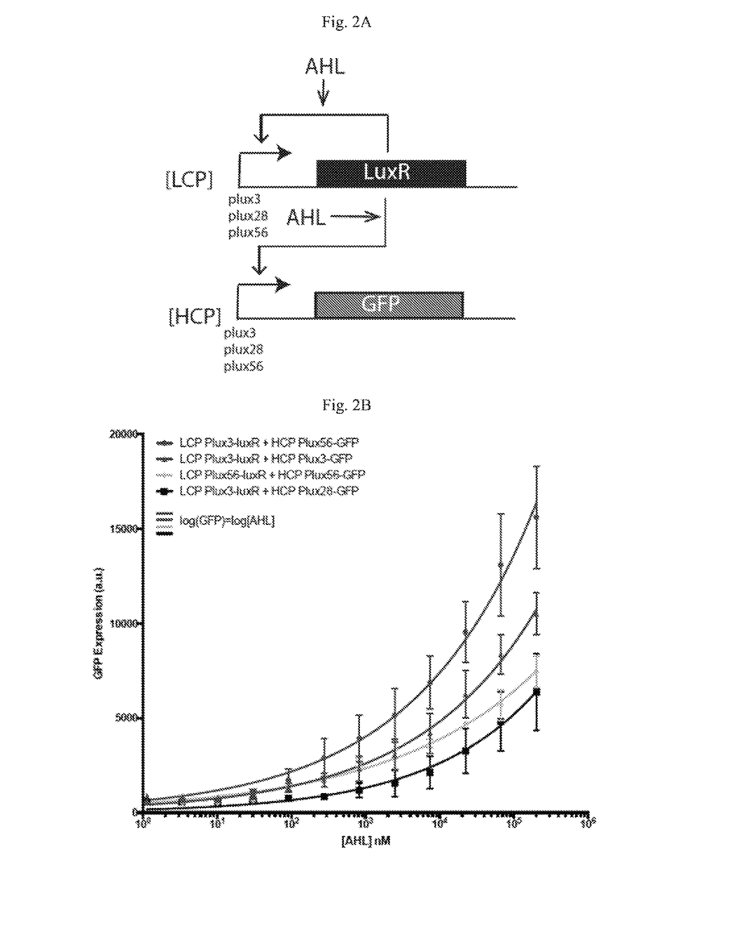

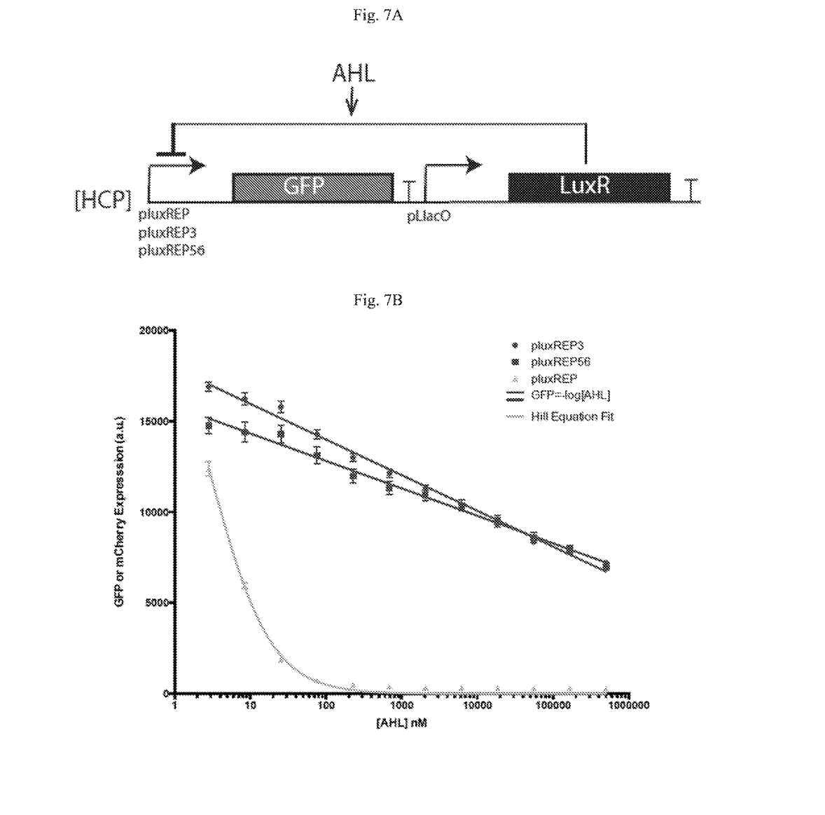

Promoters of the present disclosure may contain a (e.g., at least one) modification, relative to a wild-type (unmodified) version of the same promoter (e.g., plux3 v. pluxWT). In some embodiments, the modification alters the affinity of a regulatory protein (e.g., transcription factor) for one promoter (e.g., positive-feedback promoter) relative to another promoter (e.g., output promoter) in an analog signal processing circuit. For example, with reference to FIG. 2A, both the positive-feedback promoter and the output promoter are plux promoters, the former driving expression of LuxR and the latter driving expression of GFP. The relative affinity of each LuxR-responsive promoter for LuxR can be altered, for example, by modifying one or more nucleic acids in the lux box region of the promoter (FIG. 7).

Promoter modifications may include, for example, single or multiple nucleotide mutations (e.g., A to T, A to C, A to G, T to A, T to C, T to G, C to A, C to T, C to G, G to A, G to T, or G to C), insertions and/or deletions (relative to an unmodified promoter) in a region, or a putative region, that affects regulatory protein binding to the region. In some embodiments, a modification is in a regulatory protein (e.g., transcription factor) binding site of a promoter. A promoter may contain a single modification or more than one (e.g., 2, 3, 4, 5, 6, 7, 8, 9, 10 or more) modifications to, for example, achieve the desired binding affinity for a cognate regulatory protein.

Modified promoters having “reduced affinity” for a cognate regulatory protein may bind to the cognate regulatory protein with an affinity that is reduced by at least 5% relative to the binding affinity of the unmodified promoter to the same cognate regulatory protein. In some embodiments, a modified promoter is considered to have reduced affinity for a cognate regulatory protein if the modified promoter binds to the cognate regulatory protein with an affinity that is reduced by at least 10% to 90%, or more (e.g., at least 10%, at least 20%, at least 30%, at least 40%, at least 50%, at least 60%, at least 70%, at least 80%, at least 90%, or more) relative to the binding affinity of the unmodified promoter to the same cognate regulatory protein.

Analog signal processing circuits, in some embodiments, are designed to detect and to generate a response to one or more input signals. For example, an analog signal processing circuit may detect and generate a response to 2, 3, 4, 5, 6, 7, 8, 9 or 10 input signals. Similarly, the present disclosure provides analog signal processing circuits having multiple output molecules (e.g., 2 to 10 output molecules).

Analog signal processing circuits of the present disclosure, in some embodiments, generate a response in the form of an output molecule. An “output molecule” refers to any detectable molecule under the control of (e.g., produced in response to) an input signal. For example, as shown in FIG. 1A, GFP is an output molecule produced in response to activation of the plux promoter by AHL/LuxR. The expression level of an output protein, in some embodiments, depends on the affinity of a promoter for a particular regulatory protein. For example, the expression level of an output protein under the control of a modified promoter having reduced affinity for a regulatory protein may be less than the expression level of an output protein under the control of the unmodified promoter. Likewise, the expression level of an output protein under the control of a modified promoter having reduced affinity for a regulatory protein may be less than the expression level of an output protein under the control of a modified promoter having an even greater reduction in its affinity for the same regulatory protein.

Examples of output molecules include, without limitation, proteins and nucleic acids.

Examples of output protein molecules include, without limitation, marker proteins such as fluorescent proteins (e.g., GFP, EGFP, sfGFP, TagGFP, Turbo GFP, AcGFP, ZsGFP, Emerald, Azami green, mWasabi, T-Sapphire, EBFP, EBFP2, Azurite, mTagBFP, ECFP, mECFP, Cerulean, mTurquoise, CyPet, AmCyan1, Midori-ishi Cyan, TagCFP, mTFP1, EYFP, Topaz, Venus, mCitrine, YPET, TagYFP, PhiYFP, ZsYellow1, mBanana, Kusabira Orange, Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRedl, mRaspberry, dKeima-Tandem, HcRed-Tandem, mPlum, AQ143 and variants thereof), enzymes (e.g., catalytic enzymes such as recombinases, integrases, caspases), biosynthetic enzymes, cytokines, antibodies, regulatory proteins such as transcription factors, polymerases and chromatin remodeling factors.

Examples of output nucleic acid molecules include, without limitation, RNA interference molecules (e.g., siRNA, miRNA, shRNA), guide RNA (e.g., single-stranded guide RNA), trans-activating RNAs, riboswitches, ribozymes and RNA splicing factors.

Analog signal processing circuits may contain one or multiple (e.g., 2, 3, 4 or more) copies of an output molecule. In some embodiments, analog signal processing circuits contain two or more (e.g., 2, 3, 4 or more) differ output molecules (e.g., 2 or more different fluorescent proteins such as GFP and mCherry, or two or more different types of output molecules such as a transcription factor or small RNAs that control transcription and a fluorescent protein). In some embodiments, an output molecule regulates expression of another output molecule (e.g., is a transcription factor that regulates a promoter, which drives expression of another output molecule).

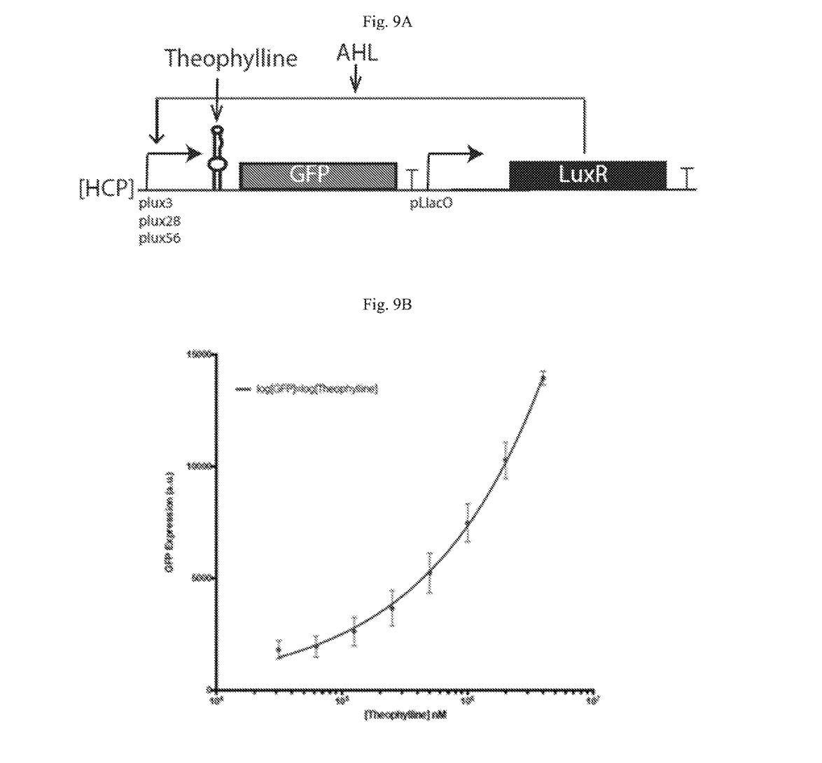

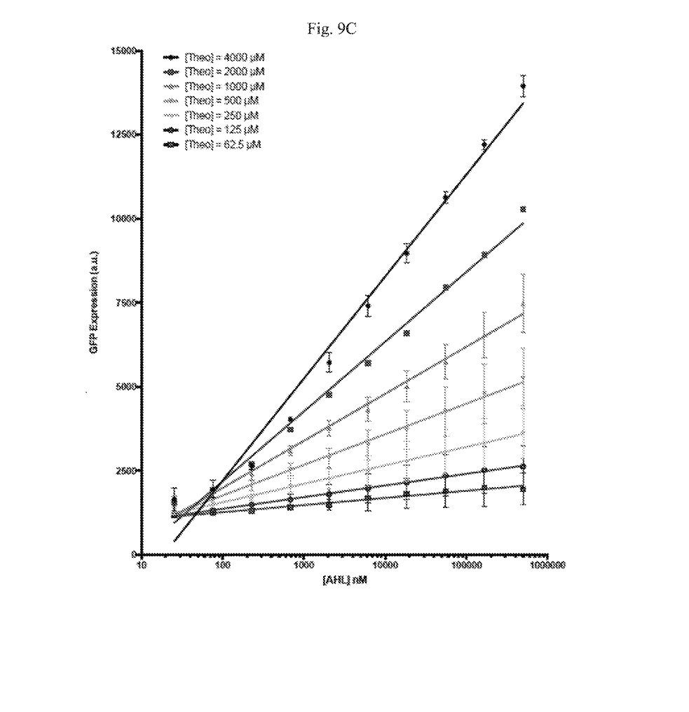

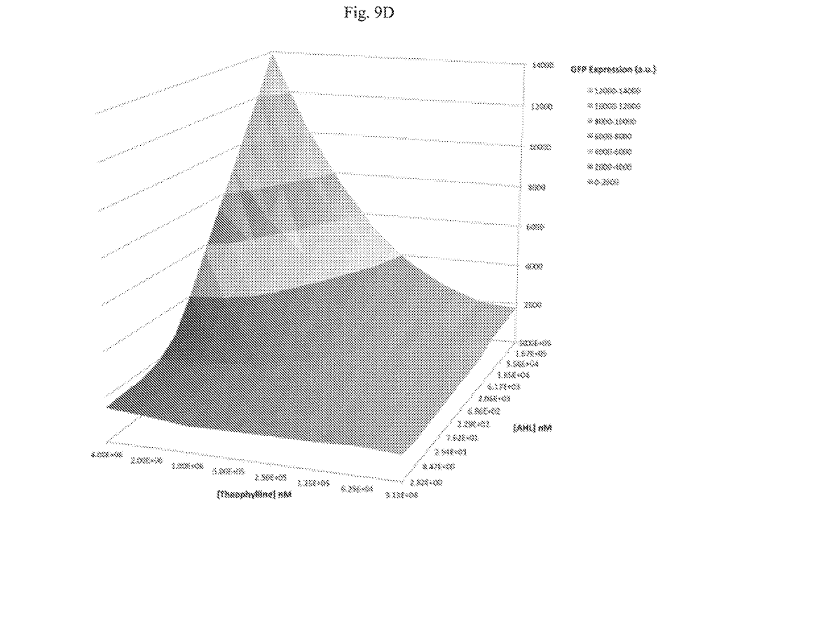

Analog signal processing circuits, and components thereof, of the present disclosure can be “tuned” by promoter modification such that the affinity of a positive-feedback promoter for a regulatory protein differs relative to the affinity of an output promoter for the same regulatory protein. Further tuning of analog signal processing circuits is contemplated herein. For example, a “regulatory sequence” may be included in a circuit to further regulate transcription, translation or degradation of an output molecule or regulatory protein. Examples of regulatory sequences as provided herein include, without limitation, ribosomal binding sites, riboswitches, ribozymes, guide RNA binding sites, microRNA binding sites, cis-repressing RNAs, siRNA binding sites and protease target sites. Regulatory sequences are typically located between a promoter and a nucleic acid to which it is operably linked such that the regulatory sequences is capable of regulating transcription and/or translation of the downstream (3′) nucleic acid and/or output molecule. Thus, in some embodiments, a regulatory sequence is located in the 5′ untranslated region (UTR) of a gene (e.g., encoding an output molecule). For example, FIG. 9A depicts a theophylline-responsive riboswitch located between a plux promoter and the downstream nucleic acid encoding GFP (e.g., in the 5′ untranslated region of the gene) to which the promoter is operably linked. In some embodiments, regulatory sequences may be located in the 3′ untranslated region of a nucleic acid and control degradation of the nucleic acid. In some embodiments, regulatory sequences may be transnationally-fused to a protein coding sequence to affect stability or intracellular-localization.

Other regulatory sequence and mechanisms are contemplated herein. For example, aptamers can be evolved in vitro to bind any molecule and then used to control a riboswitch.

In some embodiments, an analog signal processing circuit can be tuned such that a second input signal affects translation strength of an output molecule (referred to herein as an analog multiplier function). For example, a riboswitch (e.g., theophylline-responsive riboswitch) may be used to regulate translation of an output molecule. Riboswitches comprise RNA, sense their ligand in a preformed binding pocket and perform a conformational switch in response to ligand binding resulting in altered gene expression. FIG. 9A depicts an example of a theophylline-responsive riboswitch. Examples of other riboswitches for use herein below to the family class: SAM/SAM-I, SAM/SAM-II, SAM/SAM-III, TPP, purine/G, purine/A, Purine/dG, lysine or Mg²+/ykoK (as described by Edwards A L et al., Nature Education 3(9):9, 2010, incorporated by reference herein). Other riboswitches are contemplated herein.

Tuning may also be achieved by modifying (e.g., mutating) a ribosomal binding site located between a promoter and a nucleic acid to which it is operably linked.

Tuning of an analog signal processing circuit may also be achieved, for example, by controlling the level of nucleic acid expression of particular components of the circuit. This control can be achieved, for example, by controlling copy number of the nucleic acids (e.g., using low, medium and/or high copy plasmids, and/or constitutively-active promoters).

It should be understood that the “tunability” of analog signal processing circuits of the present disclosure is achieved, in some embodiments, by combining two or more tuning mechanisms as provided herein. For example, in some embodiments, analog signal processing circuits comprise a modified promoter (with reduced affinity for a regulatory protein) and a regulatory sequence (e.g., riboswitch). In some embodiments, analog signal processing circuits comprise a modified promoter and a modified ribosomal binding site. In some embodiments, analog signal processing circuits comprise a modified ribosomal binding site and regulatory sequence. Other configurations are contemplated herein.

Promoters of analog signal processing circuits may be on the same vector (e.g., plasmid) or on different vectors (e.g., each on a separate plasmid). In some embodiments, promoters may be on the same vector high copy plasmid, medium copy plasmid, or low copy plasmid.

For clarity and ease of explanation, promoters responsive to a regulatory protein (or responsive to an input signal) may be referred to as first, second or third promoters (and so on) so as to distinguish one promoter from another. It should be understood that reference to a first promoter and a second promoter, unless otherwise indicated, is intended to encompass two different promoters (e.g., pLlacO v. pluxWT). Similarly, output molecules may be referred to as a first, second or third output molecules (and so on) so as to distinguish one output molecule from another. It should be understood that reference to a first output molecule and a second output molecule, unless otherwise indicated, is encompasses two different output molecules (e.g., GFP v. mCherry).

Analog signal processing circuits of the present disclosure may be used to detect more than one input signal in a cell. For example, analog signal processing circuits may comprise two one component configured to detect one input signal and another component configured to detect another input signal, each component containing a promoter (e.g., plux v. pBAD) responsive to different regulatory proteins/input signals (e.g., LuxR/AHL v. araC/arabinose) and operably linked to different output molecules (e.g., GFP v. mCherry). In this way, an independent response to each signal may be generated.

Thus, in some embodiments, an analog signal processing circuit comprises (a) a first promoter operably linked to a nucleic acid encoding a first regulatory protein responsive to a first input signal, (b) a second promoter responsive to the regulatory protein and operably linked to a nucleic acid encoding a first output molecule, (c) a third promoter operably linked to a nucleic acid encoding a second regulatory protein responsive to a second input signal, and (d) a fourth promoter responsive to the second regulatory protein and operably linked to a nucleic acid encoding a second output molecule.

Analog signal processing circuits of the present disclosure may be expressed in a broad range of host cell types. In some embodiments, analog signal processing circuits are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, analog signal processing circuits are expressed in mammalian cells. For example, in some embodiments, analog signal processing circuits are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure are generally considered to be modified. A modified cell is a cell that contains an exogenous nucleic acid or a nucleic acid that does not occur in nature (e.g., an analog signal processing circuit of the present disclosure). In some embodiments, a modified cell contains a mutation in a genomic nucleic acid. In some embodiments, a modified cell contains an exogenous independently replicating nucleic acid (e.g., components of analog signal processing circuits present on an episomal vector). In some embodiments, a modified cell is produced by introducing a foreign or exogenous nucleic acid into a cell. Thus, provided herein are methods of introducing an analog signal processing circuit into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination).

In some embodiments, a cell contains a gene deletion.

Analog signal processing circuits of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., an analog signal processing circuit or component thereof) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

In some embodiments, provided herein are methods of delivering analog signal processing circuits (e.g., containing an analog correction component) to a subject (e.g., a human subject). Analog signal processing circuits may be delivered to subjects using, for example, in bacteriophage or phagemid vehicles, or other delivery vehicle that is capable of delivering nucleic acids to a cell in vivo. In some embodiments, analog signal processing circuits may be introduced into cells ex vivo, which cells are then delivered to a subject via injection, oral delivery, or other delivery route or vehicle.

Other uses of analog signal processing circuits are contemplated by the present disclosure. For example, the present disclosure provides cells engineered to dynamically control the synthesis of molecules or peptides based on intrinsic factors (e.g., the concentration of metabolic intermediates) or extrinsic factors (e.g., inducers); analog signal processing circuits engineered to classify a cell type (e.g., via inputs from outside of the cell, such as receptors, or inputs from inside of the cell, such as transcription factors, DNA sequence and RNAs); and cells engineered to synthesize materials in a spatial pattern based on, for example, environmental cues.

It should be understood that while analog signal processing circuits of the present disclosure, in many embodiments, are delivered to cells or are otherwise used in vivo, the present disclosure is not so limited. Analog signal processing circuits as provided herein may be used in vivo or in vitro, intracellularly or extracellularly (e.g., using cell-free extracts/lysates). For example, analog signal processing circuits may be used in an in vitro abiotic paper-based platform as described in Pardee K et al. (Cell, Corrected Proof published online Oct. 23, 2014, in press, incorporated by reference herein) to, for example, enable rapid prototyping for cell-based research and gene circuit design.

The present disclosure also provides aspects encompassed by the following numbered paragraphs:

1. An analog signal processing circuit comprising:

(a) a first promoter operably linked to a nucleic acid encoding a regulatory protein responsive to an input signal; and

(b) a second promoter responsive to the regulatory protein and operably linked to a nucleic acid encoding an output molecule.

2. The circuit of paragraph 1, wherein the promoter of (a) is a constitutively-active promoter.

3. The circuit of paragraph 1, wherein the promoter of (a) is responsive to the regulatory protein.

4. The circuit of paragraph 3, wherein the promoter of (a) comprises a modification that alters the binding affinity of the regulatory protein for the promoter of (a), relative to a similar unmodified promoter.

5. The circuit of paragraph 4, wherein the modification is a nucleic acid mutation.

6. The circuit of any one of paragraphs 1-5, wherein the promoter of (b) comprises a modification that alters the binding affinity of the regulatory protein for the promoter of (b), relative to a similar unmodified promoter.

7. The circuit of paragraph 6, wherein the modification is a nucleic acid mutation.

8. The circuit of any one of paragraphs 1-7, wherein (a) and (b) are on the same vector.

9. The circuit of paragraph 8, wherein the vector is a low copy plasmid, a medium copy plasmid or a high copy plasmid.

10. The circuit of any one of paragraphs 1-9, wherein the promoter of (b) is activated when bound by the regulatory protein.

11. The circuit of any one of paragraphs 1-10, wherein the promoter of (b) is repressed when bound by the regulatory protein.

12. The circuit of any one of paragraphs 1-11, wherein (b) further comprises a regulatory sequence that regulates production of the output molecule and is located between the second promoter and the nucleic acid encoding the output molecule.

13. The circuit of paragraph 12, wherein the regulatory sequence regulates transcription or translation of the output molecule.

14. The circuit of paragraph 12 or 13, wherein the regulatory sequence is a ribosomal binding site.

15. The circuit of paragraph 12 or 13, wherein the regulatory sequence is a modified ribosomal binding site comprising a modification that alters the binding affinity of a ribosome for the modified ribosomal binding site, relative to a similar unmodified ribosomal binding site.

16. The circuit of paragraph 12 or 13, wherein the regulatory sequence is a riboswitch.

17. The circuit of paragraph 16, wherein the riboswitch is responsive to theophylline.

18. The circuit of paragraph 1, wherein the promoter of (b) is a plux promoter that comprises a modification that alters the binding affinity of LuxR for the plux promoter of (b), relative to a similar unmodified promoter.

19. The circuit of 18, wherein the promoter of (a) is operably linked to a nucleic acid encoding a LuxR protein.

20. The circuit of paragraph 18 or 19, wherein the promoter of (a) is a constitutively-active promoter.

21. The circuit of paragraph 18 or 19, wherein the promoter of (a) is a plux promoter.

22. The circuit of paragraph 21, wherein the plux promoter of (a) comprises a modification that alters the binding affinity of LuxR for the plux promoter of (a), relative to a similar unmodified promoter.

23. The circuit of paragraph 1, wherein the promoter of (b) is a pBAD promoter that comprises a modification that alters the binding affinity of araC for the pBAD promoter of (b), relative to a similar unmodified promoter.

24. The circuit of paragraph 23, wherein the promoter of (a) is operably linked to a nucleic acid encoding an araC protein.

25. The circuit of paragraph 23 or 24, wherein the promoter of (a) is a constitutively-active promoter.

26. The circuit of paragraph 23 or 24, wherein the promoter of (a) is a pBAD promoter.

27. The circuit of paragraph 26, wherein the pBAD promoter of (a) comprises a modification that alters the binding affinity of araC for the pBAD promoter of (a), relative to a similar unmodified promoter.

28. The circuit of any one of paragraphs 1-27, wherein the output molecule of (b) is a fluorescent output molecule.

29. A cell or cell lysate comprising the circuit of any one of paragraphs 1-28.

30. The cell or cell lysate of paragraph 29, wherein the cell is a bacterial cell.

31. The cell or cell lysate of paragraph 30, wherein the bacterial cell is an Escherichia coli cell.

32. The cell or cell lysate of any one of paragraphs 29-31 further comprising the input signal.

33. The cell or cell lysate of paragraph 32, wherein the input signal modulates activity of the of the regulatory protein.

34. The cell or cell lysate of paragraph 33, wherein the input signal activates the regulatory protein.

35. The cell or cell lysate of any one of paragraphs 32-34, wherein the input signal is a chemical input signal.

36. A method of analog signal processing in cells, comprising: providing a cell or cell lysate that comprises the circuit of any one of paragraphs 1-28; contacting the cell with an input signal that modulates the regulatory protein; and detecting in the cell or cell lysate an expression level of the output molecule.

37. The method of paragraph 36 further comprising contacting the cell or cell lysate with different concentrations of the input signal.

38. The method of paragraph 36 or 37 further comprising quantifying levels of the output molecule.

39. The method of any one of paragraphs 36-38, wherein the cell is a bacterial cell.

40. The method of paragraph 39, wherein the bacterial cell is an Escherichia coli cell.

41. An analog signal processing circuit comprising: (a) a first constitutively-active promoter operably linked to a nucleic acid encoding a regulatory protein responsive to an input signal; and (b) a second promoter responsive to the regulatory protein and operably linked to a nucleic acid encoding an output molecule, wherein the second promoter comprises a modification that alters the binding affinity of the regulatory protein for the second promoter, relative to a similar unmodified promoter.

42. An analog signal processing circuit comprising: (a) a first promoter operably linked to a nucleic acid encoding a regulatory protein responsive to an input signal, wherein the first promoter is responsive to the regulatory protein and comprises a modification that alters the binding affinity of the regulatory protein for the first promoter; and (b) a second promoter responsive to the regulatory protein and operably linked to a nucleic acid encoding an output molecule.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

### Example 1—Altering Transcription Factor Binding Affinity in a Positive-Feedback-Configured Circuit Yields a Wide-Dynamic Range of Behavior

The purpose of this study is to show that altering the affinity of a transcription factor for its cognate binding site in a promoter operably linked to an output molecule in a positive-feedback configuration yields a wide-dynamic range of behavior. In this Example, analog signal processing circuits were configured as positive-feedback loops to contain two components: the first containing a wild-type plux promoter or one of three different mutated plux promoters driving expression of LuxR protein on a low copy plasmid, and the second containing one of three different mutated plux promoters driving expression of green fluorescent protein (GFP) on a high copy plasmid (FIGS. 1A and 2A). Each of the three mutated plux promoters—plux3, plux28 and plux56—have a reduced affinity for LuxR, relative to the wild-type plux promoter. The components of the circuits were delivered to Escherichia coli (E. coli) cells (see Methods), and then the cells were exposed to various concentrations of input signal (or inducer), acyl-homoserine lactone (AHL). A measure of GFP fluorescence (FIGS. 1B and 2B) shows that mutated promoters confer on the circuits a wide-dynamic range of behavior in response to various concentrations of AHL, without fusion to GFP, on two different plasmids, in a positive-feedback configuration.

### Example 2—Altering Transcription Factor Binding Affinity in an Open-Loop-Configured Circuit Yields a Wide-Dynamic Range of Behavior

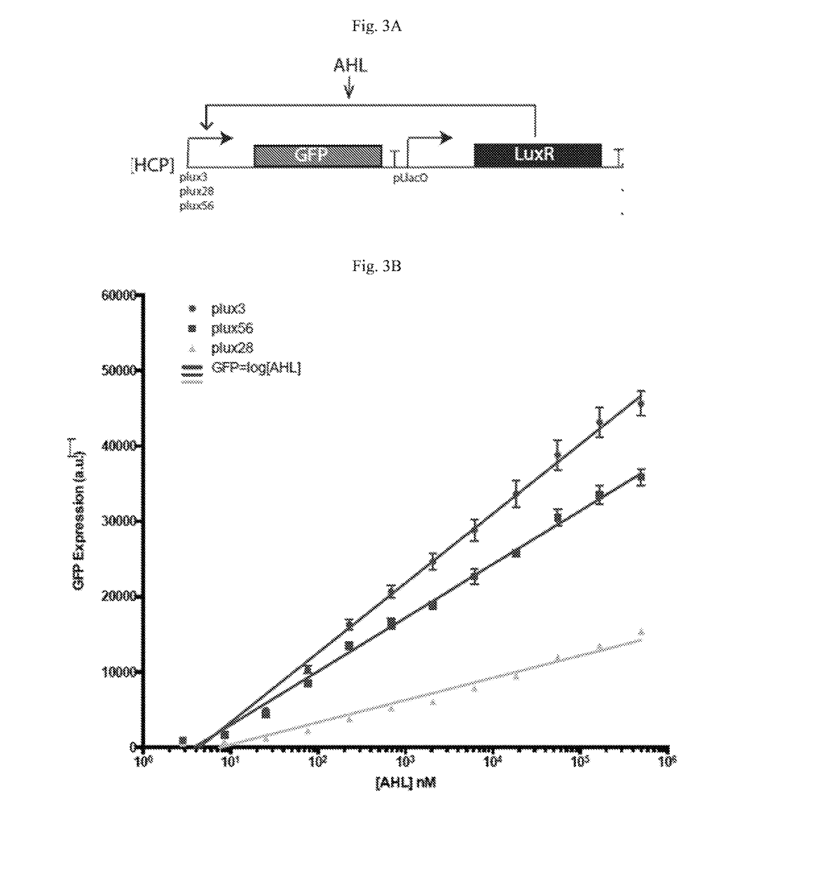

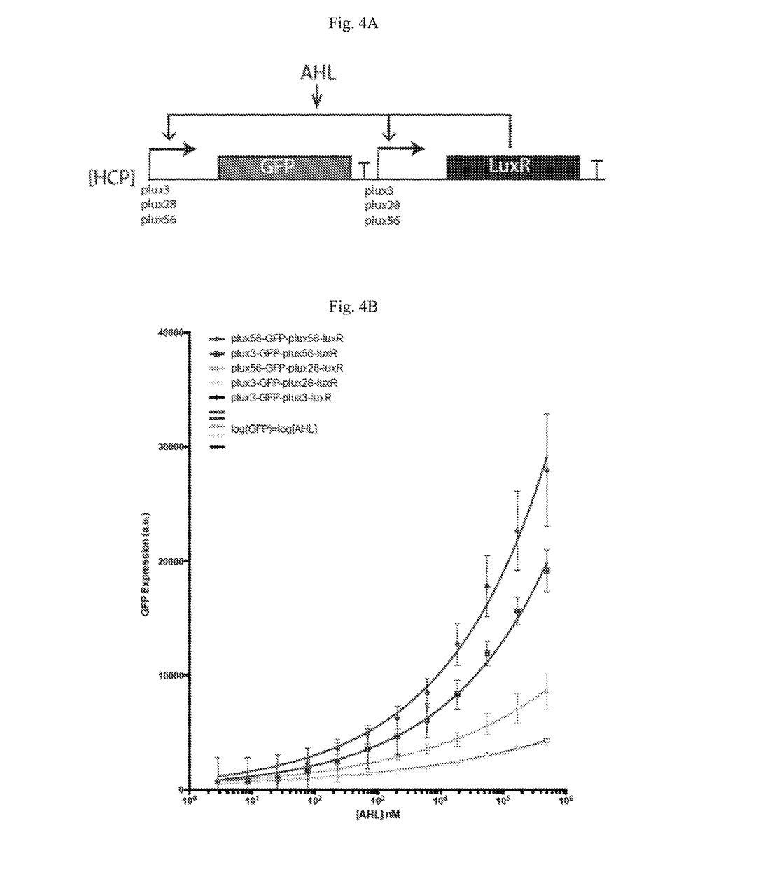

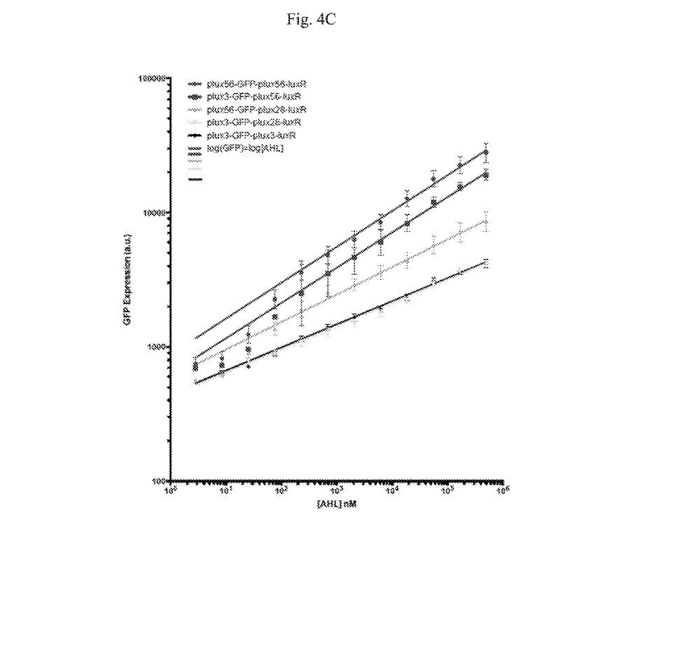

The purpose of this study is to show that altering the affinity of a transcription factor for its cognate binding site in a promoter operably linked to an output molecule in an open-loop configuration yields a wide-dynamic range of behavior. In this Example, analog signal processing circuits were configured as open loops to contain a single component: one of three different mutated plux promoters driving expression of GFP and a constitutively-active pLlacO promoter or one of three different mutated plux promoters driving expression of LuxR protein on a high copy plasmid (FIG. 3A and FIG. 4A). The circuits were delivered to E. coli cells (see Methods), and then the cells were exposed to various concentrations of AHL. A measure of GFP fluorescence (FIG. 3B and FIG. 4B) shows that mutated promoters confer on the circuits a wide-dynamic range of behavior in response to various concentrations of AHL, with fusion to GFP on the same high copy plasmid, in an open-loop configuration.

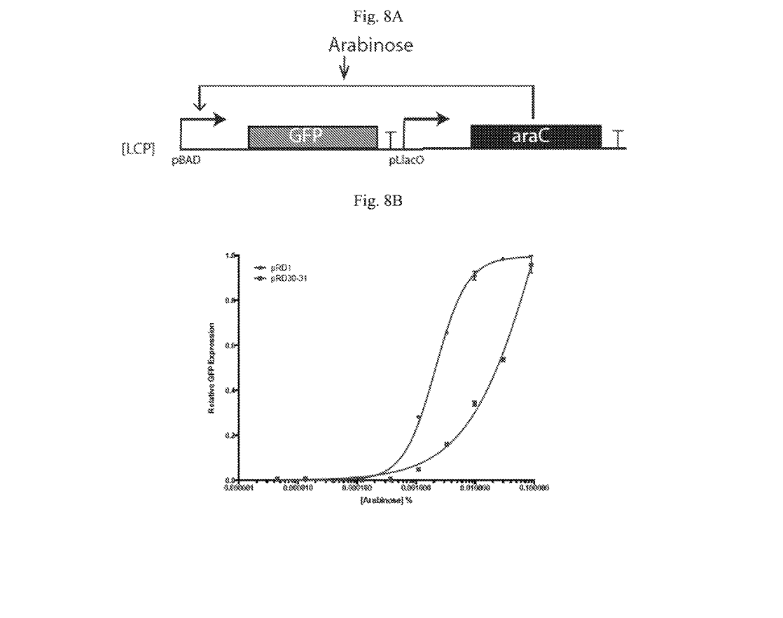

Another set of analog signal processing circuits were configured as open loops to contain a single component: a truncated pBAD promoter with a wild-type binding site for araC protein or one of two different truncated pBAD promoters with mutated binding sites for araC protein driving expression of GFP and a constitutively-active pLlacO promoter driving expression of araC protein on a low copy plasmid (FIG. 8A). The circuits were delivered to E. coli cells (see Methods), and then the cells were exposed to various concentrations of arabinose. A measure of GFP fluorescence (FIG. 8B) shows that mutated promoters confer on the circuits a wide-dynamic range of behavior in response to various concentrations of arabinose, with fusion to GFP, on the same low copy plasmid, in an open-loop configuration.

### Example 3—a Comparison of Analog Signal Processing Circuit Configurations

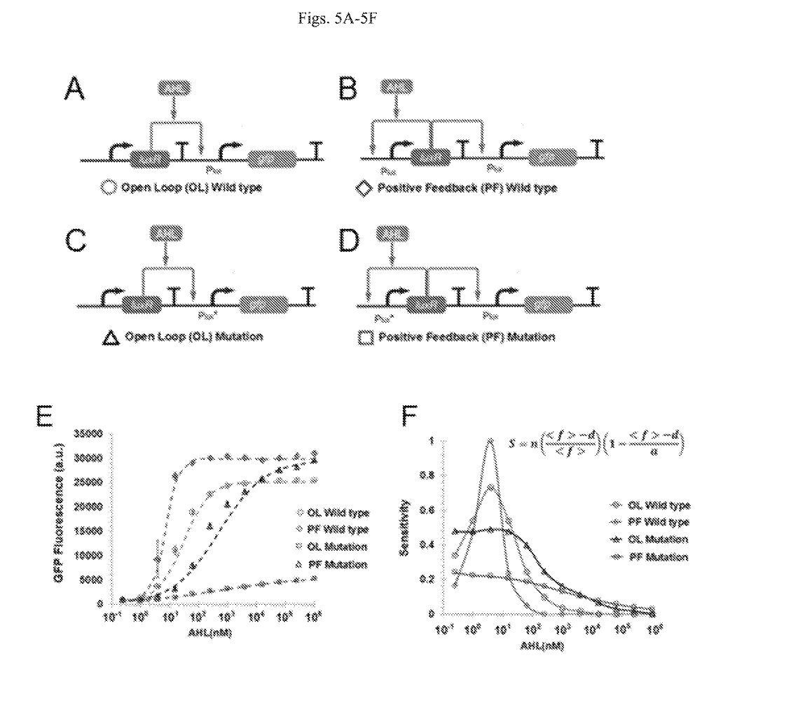

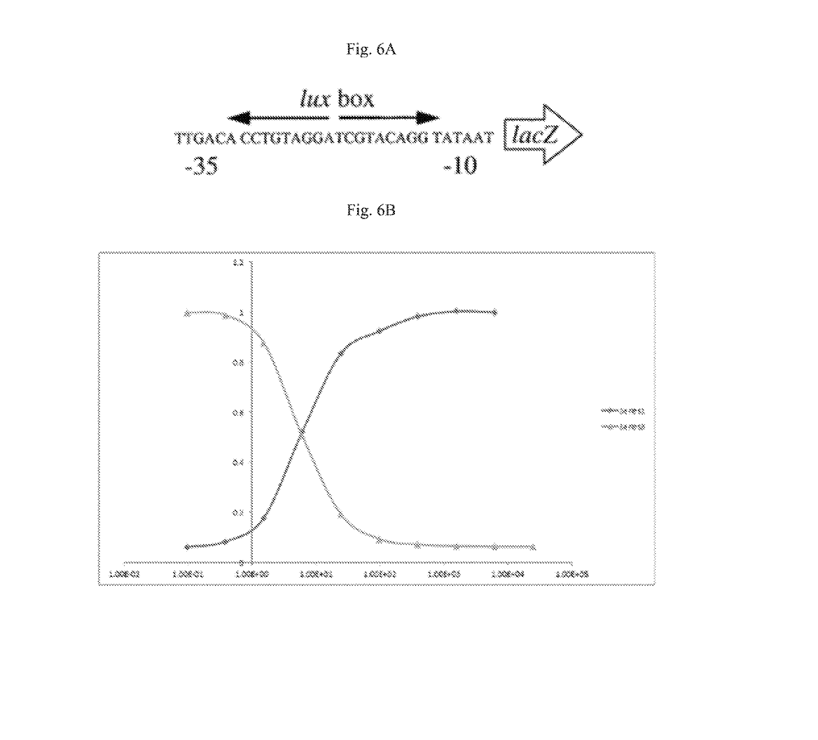

The purpose of this study is to show a comparison of several analog signal processing circuit configurations. In this Example, analog signal processing circuits were configured as follows: an open-loop configuration with a constitutively-active promoter driving expression of LuxR and a wild-type plux promoter driving expression of GFP (FIG. 5A); a positive-feedback configuration with a wild-type plux promoter driving expression of LuxR and a wild-type plux promoter driving expression of GFP (FIG. 5B); an open-loop configuration with a constitutively-active promoter driving expression of LuxR and a mutated plux promoter driving expression of GFP (FIG. 5C); and a positive-feedback configuration with a mutated plux promoter driving expression of LuxR and a wild-type plux promoter driving expression of GFP (FIG. 5D). The circuits were delivered to E. coli cells (see Methods), and then the cells were exposed to various concentrations of AHL. A measure of GFP fluorescence (FIG. 5E) shows that the circuits without mutated promoters (shown in FIGS. 5A and 5B), yield digital-like signal processing behavior, and the circuits with mutated promoters (shown in FIGS. 5C and 5D), yield analog-like signal processing behavior. FIG. 5F shows the sensitivity of circuits in FIG. 5A-5D calculated from the GFP expression data in FIG. 5E.

### Example 4—Altering Transcription Factor Binding Affinity of a Repressor in an Open-Loop-Configured Circuit Yields a Wide-Dynamic Range of Behavior

The purpose of this study is to show that a mutation within a repressor location of the plux promoter yields a negative-slope wide-dynamic range of behavior. In this Example, analog signal processing circuits were configured as open loops to contain a single component: a plux promoter with a repressor location (pluxREP), or one of two different mutated plux promoters (pluxREP3, or pluxREP56) driving expression of GFP and a constitutively-active pLlacO promoter driving expression of LuxR protein on a high copy plasmid (FIG. 7A). The circuits were delivered to E. coli cells (see Methods), and then the cells were exposed to various concentrations of AHL. A measure of GFP fluorescence (FIG. 7B) shows that a mutation within a repressor location of the plux promoter yields a negative-slope wide-dynamic range of behavior. Without the mutation, the promoter (pluxREP) yields a negative-slope with “digital” behavior.

### Example 5—Altering Transcription Factor Binding Affinity in Series with a Riboswitch in an Open-Loop-Configured Circuit Yields a Wide-Dynamic Range of Behavior

The purpose of this study is to show that altering the affinity of a transcription factor for its cognate binding site in a promoter operably linked to an output molecule together with post-transcriptional/translation regulation of the output molecule, in an open-loop configuration, yields a wide-dynamic range of behavior. In this Example, analog signal processing circuits were configured as open loops to contain a single component: one of three different mutated plux promoters driving expression of GFP with a riboswitch (responsive to theophylline) located in the 5′ untranslated region (UTR) of the GFP and a constitutively-active pLlacO promoter driving expression of LuxR protein on a high copy plasmid (FIG. 9A). The circuits were delivered to E. coli cells (see Methods), and then the cells were exposed to various concentrations of theophylline and either a constant concentration of AHL (FIG. 9B) or varying concentrations of AHL (FIG. 9C). A measure of GFP fluorescence (FIGS. 9A and 9B) shows that mutated promoters in series with a riboswitch confer on the circuits a wide-dynamic range of behavior in response to two different input signals (theophylline and AHL).

### Methods

Overnight cultures of E. coli strains were at 37° C., in a VWR 1585 shaking incubator at 300 r.p.m., in Luria-Bertani-Miller medium (Fisher) with appropriate antibiotics. Inducers/input signals used were arabinose, AHL 3OC6HSL, and Theophylline (Sigma-Aldrich). Over-night cultures were diluted 1:100 into fresh Luria-Bertani medium and antibiotics, and were incubated at 37° C. and 300 r.p.m. for 20 min. Cultures were then moved into 96-well plates, combined with inducers and incubated for 4 hours, 20 min in a microplate shaker (37° C., 700 r.p.m.), until they had an attenuance of D600 nm, 0.6-0.8.

Cells were then diluted four-fold into a new 96-well plate containing fresh PBS and immediately assayed using a BD LSRFortessa high-throughput sampler. At least 50,000 events were recorded in all experiments, and these data were then gated by forward scatter and side scatter using FloJo software. The geometric means of the gated fluorescence distributions were calculated using FloJo.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An analog signal processing circuit comprising:
(a) a first promoter operably linked to a nucleic acid encoding a regulatory protein responsive to an input signal; and
(b) a second promoter responsive to the regulatory protein and operably linked to a nucleic acid encoding an output molecule.

2. The circuit of claim 1, wherein the promoter of (a) is a constitutively-active promoter.

3. The circuit of claim 1, wherein the promoter of (a) is responsive to the regulatory protein.

4. The circuit of claim 3, wherein the promoter of (a) comprises a modification that alters the binding affinity of the regulatory protein for the promoter of (a), relative to a similar unmodified promoter.

5. The circuit of claim 4, wherein the modification is a nucleic acid mutation.

6. The circuit of claim 1, wherein the promoter of (b) comprises a modification that alters the binding affinity of the regulatory protein for the promoter of (b), relative to a similar unmodified promoter.

7. The circuit of claim 6, wherein the modification is a nucleic acid mutation.

8. The circuit of claim 1, wherein (a) and (b) are on the same vector.

9. The circuit of claim 8, wherein the vector is a low copy plasmid, a medium copy plasmid or a high copy plasmid.

10. The circuit of claim 1, wherein the promoter of (b) is activated when bound by the regulatory protein.

11. The circuit of claim 1, wherein the promoter of (b) is repressed when bound by the regulatory protein.

12. The circuit of claim 1, wherein (b) further comprises a regulatory sequence that regulates production of the output molecule and is located between the second promoter and the nucleic acid encoding the output molecule.

13. The circuit of claim 12, wherein the regulatory sequence regulates transcription or translation of the output molecule.

14. The circuit of claim 12, wherein the regulatory sequence is a ribosomal binding site.

15. The circuit of claim 12, wherein the regulatory sequence is a modified ribosomal binding site comprising a modification that alters the binding affinity of a ribosome for the modified ribosomal binding site, relative to a similar unmodified ribosomal binding site.

16. The circuit of claim 12, wherein the regulatory sequence is a riboswitch.

17. The circuit of claim 16, wherein the riboswitch is responsive to theophylline.

18. The circuit of claim 1, wherein the promoter of (b) is a plux promoter that comprises a modification that alters the binding affinity of LuxR for the plux promoter of (b), relative to a similar unmodified promoter.

19. The circuit of 18, wherein the promoter of (a) is operably linked to a nucleic acid encoding a LuxR protein.

20. The circuit of claim 18, wherein the promoter of (a) is a constitutively-active promoter.

21. The circuit of claim 18, wherein the promoter of (a) is a plux promoter.

22. The circuit of claim 21, wherein the plux promoter of (a) comprises a modification that alters the binding affinity of LuxR for the plux promoter of (a), relative to a similar unmodified promoter.

23. The circuit of claim 1, wherein the promoter of (b) is a pBAD promoter that comprises a modification that alters the binding affinity of araC for the pBAD promoter of (b), relative to a similar unmodified promoter.

24. The circuit of claim 23, wherein the promoter of (a) is operably linked to a nucleic acid encoding an araC protein.

25. The circuit of claim 23, wherein the promoter of (a) is a constitutively-active promoter.

26. The circuit of claim 23, wherein the promoter of (a) is a pBAD promoter.

27. The circuit of claim 26, wherein the pBAD promoter of (a) comprises a modification that alters the binding affinity of araC for the pBAD promoter of (a), relative to a similar unmodified promoter.

28. The circuit of claim 1, wherein the output molecule of (b) is a fluorescent output molecule.

29. A cell or cell lysate comprising the circuit of claim 1.

30. The cell or cell lysate of claim 29, wherein the cell is a bacterial cell.

31. The cell or cell lysate of claim 30, wherein the bacterial cell is an Escherichia coli cell.

32. The cell or cell lysate of claim 29 further comprising the input signal.

33. The cell or cell lysate of claim 32, wherein the input signal modulates activity of the of the regulatory protein.

34. The cell or cell lysate of claim 33, wherein the input signal activates the regulatory protein.

35. The cell or cell lysate of claim 32, wherein the input signal is a chemical input signal.

36. A method of analog signal processing in cells, comprising:
providing a cell or cell lysate that comprises the circuit of claim 1;
contacting the cell with an input signal that modulates the regulatory protein; and
detecting in the cell or cell lysate an expression level of the output molecule.

37. The method of claim 36 further comprising contacting the cell or cell lysate with different concentrations of the input signal.

38. The method of claim 36 further comprising quantifying levels of the output molecule.

39. The method of claim 36, wherein the cell is a bacterial cell.

40. The method of claim 39, wherein the bacterial cell is an Escherichia coli cell.

41. An analog signal processing circuit comprising:
(a) a first constitutively-active promoter operably linked to a nucleic acid encoding a regulatory protein responsive to an input signal; and
(b) a second promoter responsive to the regulatory protein and operably linked to a nucleic acid encoding an output molecule, wherein the second promoter comprises a modification that alters the binding affinity of the regulatory protein for the second promoter, relative to a similar unmodified promoter.

42. An analog signal processing circuit comprising:
(a) a first promoter operably linked to a nucleic acid encoding a regulatory protein responsive to an input signal, wherein the first promoter is responsive to the regulatory protein and comprises a modification that alters the binding affinity of the regulatory protein for the first promoter; and
(b) a second promoter responsive to the regulatory protein and operably linked to a nucleic acid encoding an output molecule.
